(19) United States
(12) Patent Application Publication
(10) Pub. No.: US 2008/0091023 A1
(43) Pub. Date: Apr. 17, 2008

(54) Processes for optical resolution of 1-phenyl-1,2,3,4-tetrahydroisoquinoline

(76) Inventors: Nurit Perlman, Kfar Saba (IL); Tamar Nidam, Yehud (IL)

Correspondence Address: KENYON & KENYON LLP, ONE BROADWAY, NEW YORK, NY, 10004, US

(21) Appl. No.: 11/890,289

(22) Filed: Aug. 3, 2007

Related U.S. Application Data
(60) Provisional application No. 60/835,806, filed on Aug. 3, 2006.
(60) Provisional application No. 60/845,260, filed on Sep. 18, 2006.
(60) Provisional application No. 60/845,261, filed on Sep. 18, 2006.
(60) Provisional application No. 60/859,951, filed on Nov. 20, 2006.
(60) Provisional application No. 60/859,952, filed on Nov. 20, 2006.
(60) Provisional application No. 60/878,913, filed on Jan. 4, 2007.
(60) Provisional application No. 60/898,789, filed on Jan. 31, 2007.
(60) Provisional application No. 60/898,888, filed on Jan. 31, 2007.
(60) Provisional application No. 60/930,391, filed on May 15, 2007.
(60) Provisional application No. 60/949,112, filed on Jul. 11, 2007.

(51) Int. Cl. C07D 217/02; C07D 401/14

(52) U.S. Cl. 546/137; 546/144

(57) ABSTRACT

Optically pure 1(S)-phenyl-1,2,3,4-tetrahydroisoquinoline tartrate is prepared. The 1(S)-phenyl-1,2,3,4-tetrahydroisoquinoline tartrate is particularly useful for preparing solifenacin succinate.

### CROSS-REFERENCE TO RELATED APPLICATIONS

This application claims the benefit of Provisional Application Ser. No. 60/835,806, filed Aug. 3, 2006, Provisional Application Ser. No. 60/845,260, filed Sep. 18, 2006, Provisional Application Ser. No. 60/845,261, filed Sep. 18, 2006, Provisional Application Ser. No. 60/859,951, filed Nov. 20, 2006, Provisional Application Ser. No. 60/859,952, filed Nov. 20, 2006, Provisional Application Ser. No. 60/878,913, filed Jan. 4, 2007, Provisional Application Ser. No. 60/898,789, filed Jan. 31, 2007, Provisional Application Ser. No. 60/898,888, filed Jan. 31, 2007, Provisional Application Ser. No. 60/930,391, filed May 15, 2007, and to Provisional Application Ser. No. 60/949,112, filed Jul. 11, 2007. The contents of these applications are incorporated herein in their entirety by reference.

### FIELD OF INVENTION

The present invention relates to optical resolution processes for 1-phenyl-1,2,3,4-tetrahydroisoquinoline, which is a useful intermediate for making solifenacin.

### BACKGROUND OF THE INVENTION

(3R)- 1-Azabicyclo[2.2.2]oct-3-yl-(1S)-1-phenyl-3,4-dihydroisoquinoline-2-(1H)-carboxylate [(1S)-1-Phenyl-1,2,3,4-tetrahydroisoquinoline-2-carboxylic acid 3(R)-quinuclidinyl ester] is known as solifenacin, YM-905 (in its free base form) and YM-67905 (in its succinate form). Solifenacin has the molecular formula C₂₃H₂₆O₂, a molecular weight of 362.4647, and the following chemical structure:

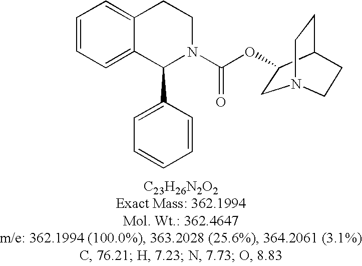

Solifenacin succinate is a urinary antispasmodic, acting as a selective antagonist to the M(3)-receptor. It is used as treatment of symptoms of overactive bladder, such as urinary urgency and increased urinary frequency as may occur in patients with overactive bladder syndrome (“OAB”), as reviewed in Chilman-Blair, Kim et al. Drugs of Today 40(4):343-353 (2004). Its crystalline powder is white to pale yellowish-white and is freely soluble at room temperature in water, glacial acetic acid, dimethylsulfoxide (“DMSO”), and methanol.

The commercial tablet is marketed under the name Vesicare®. Vesicare® has been approved by the FDA for once daily treatment of OAB and is available in 5 mg and 10 mg tablets.

U.S. Pat. Nos. 6,017,927 (“the '927 patent”) and 6,174,896 (“the '896 patent”) disclose compounds of general formula: 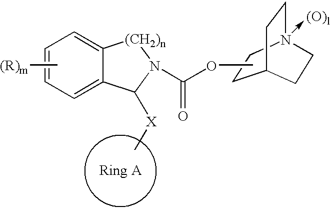
,which is reported to encompass solifenacin and its salts. The '927 patent is listed in the FDA's Orange Book for Vesicare®.

PCT Publication Nos. WO 2005/087231, WO 2005/75474, and WO 2005/105795 more specifically reported to encompass processes for the production of solifenacin and its salt to a high degree of purity for medicinal use.

Two processes for synthesizing solifenacin that have been described use the following as key starting materials: (R)-(−)quinuclidinol and 1-phenyl-1,2,3,4-tetrahydroisoquinoline (“IQL”).

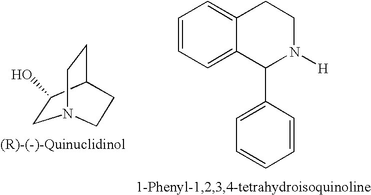

The quinuclidinol reactant is available commercially.

The overall synthesis as reported by Mealy, N et al. in Drugs of the Future 24 (8): 871-874 (1999) is depicted in Scheme 1:

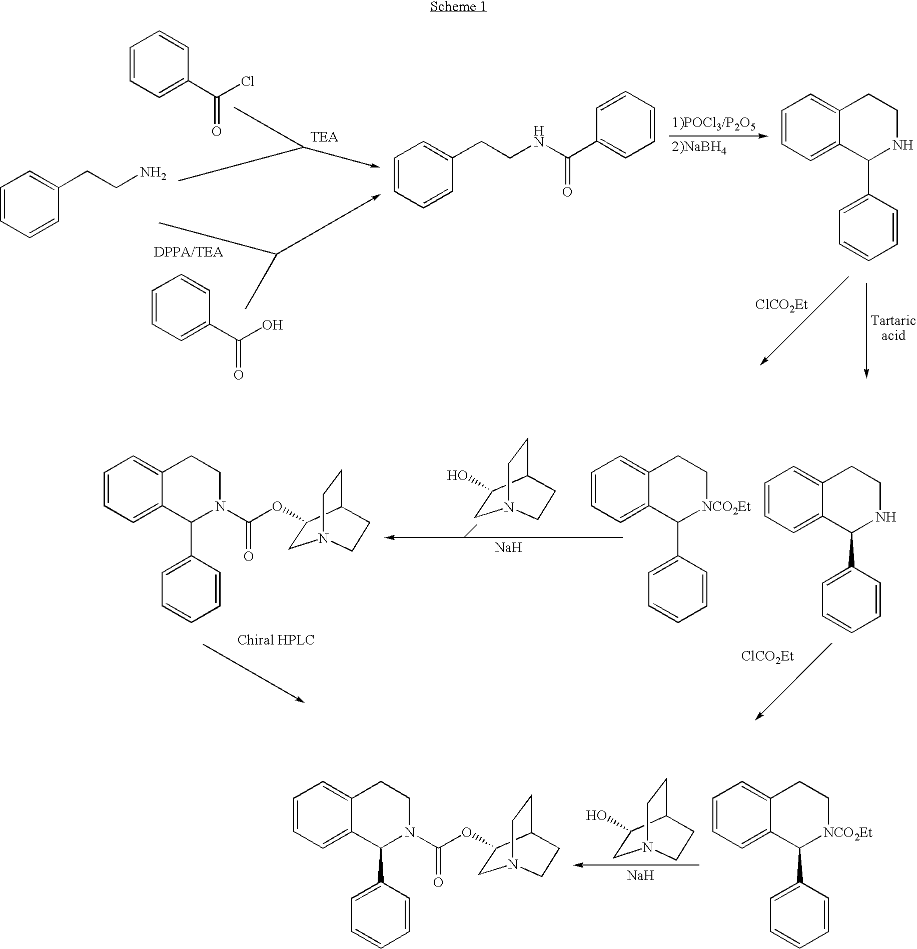

The '927 patent reported to encompass another process for the preparation of solifenacin, wherein 3-quinuclidinyl chloroformate monohydrochloride is admixed with 1(S)-phenyl-1,2,3,4-tetrahydroisoquinoline (“(S)-IQL”) to obtain solifenacin, as shown below in Scheme 2:

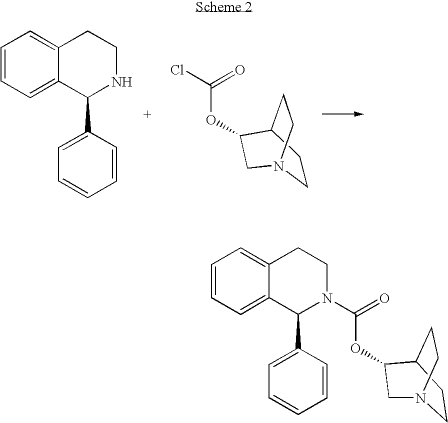

Thus, in a number of processes for the synthesis of solifenacin, (S)-IQL is a key intermediate. Optical resolution of this intermediate is disclosed in Monatshefte fur chemie, vol. 53-54: 956-962 (1929). The procedure involves addition of a solution of (D)-tartaric acid in water to the free base. Water distillation proceeds until a syrup is obtained and precipitation has occurred. The crystals are recrystallized four times from water. Naito et al. in J. Med. Chem. 48(21): 6597-6606 (2005) discloses a similar method using ethanol for addition of tartaric and recrystallization from water. These processes all involve multiple steps of crystallization.

There is a need in the art for new processes for the optical resolution of IQL, that are less time consuming, and thus applicable for industrial process.

### BRIEF SUMMARY OF THE INVENTION

The present invention provides a process for the optical resolution of IQL by preparing (S)-1-phenyl-1,2,3,4-tetrahydroisoquinoline tartrate (“(S)-IQL tartrate”). (S)-IQL tartrate may be prepared by a process combining IQL, (D)-tartaric acid, and an organic solvent selected from IPA and EtOAc.

(S)-IQL tartrate may also be prepared by a process comprising (a) combining 1-phenyl-1,2,3,4-tetrahydroisoquinoline oxalate (“IQL oxalate”), water, an organic solvent selected from THF and EtOAc, and an inorganic base to obtain (S)-IQL; and (b) combining the (S)-IQL from step (a) with (D)-tartaric acid to obtain (S)-IQL tartrate.

The present invention provides processes for preparing (S)-IQL tartrate with an enantiomeric purity of at least about 90%, preferably at least about 95%, more preferably at least about 98%.

The present invention provides a process for preparing solifenacin succinate by preparing (S)-IQL tartrate and converting (S)-IQL tartrate to solifenacin succinate.

### DETAILED DESCRIPTION OF THE INVENTION

As used herein, the term “room temperature” or “RT” refers the ambient temperature of a typical laboratory, which is usually about 15° C. to about 30° C., often about 18° C. to about 25° C.

As used herein, the term “reflux temperature” refers to the boiling point of the mixture being heated.

As used herein, the term “vacuum” refers to a pressure of about to 2 mmHg to about 100 mmHg.

As used herein, the term “(S)-IQL” refers to 1(S)-phenyl-1,2,3,4-tetrahydroisoquinoline, the term “(R)-IQL” refers to 1(R)-phenyl-1,2,3,4-tetrahydroisoquinoline, the term “IQL” refers to 1-phenyl-1,2,3,4-tetrahydroisoquinoline or a mixture of (S)-IQL and (R)-IQL with low optical purity (e.g., a racemate), the term “(S)-IQL tartrate” refers to 1(S)-phenyl-1,2,3,4-tetrahydroisoquinoline tartrate, the term “IQL tartrate” refers to 1-phenyl- 1,2,3,4-tetrahydroisoquinoline tartrate, and the term “IQL oxalate” refers to 1-phenyl- 1,2,3,4-tetrahydroisoquinoline oxalate.

As used herein, the term “enantiomeric purity” refers to the purity of one enantiomer with respect to the other enantiomer.

As used herein, the term “DMSO” refers to dimethylsulfoxide, the term “EPA” refers to isopropyl alcohol, the term “EtOAc” refers to ethyl acetate, the term “THF” refers to tetrahydrofuran, and the term “EtOH” refers to ethanol.

The present invention preferably encompasses processes for optical resolution of IQL. These processes may be suitable for industrial production. Preferably, the processes do not involve distillation operations or time-consuming crystallization steps.

The invention encompasses a process for the optical resolution of IQL by preparing (S)-IQL tartrate. (S)-IQL tartrate may be prepared by a process comprising combining IQL, (D)-tartaric acid, and an organic solvent selected from IPA and EtOAc. Optionally, water is added.

In one embodiment, the process comprises (a) combining the 1-phenyl-1,2,3,4-tetrahydroisoquinoline and organic solvent with water to form a first mixture; and (b) combining (D)-tartaric acid with the first mixture of step (a) to form a second mixture.

Optionally, the process further comprises a heating step before and/or after the (D)-tartaric acid is added. Preferably, the heating is to a temperature of about 40° C. to about reflux temperature, more preferably to a temperature of about 40° C. to about 65° C. Preferably, the heating of the first mixture takes place at a sufficient temperature for a sufficient time to obtain a solution. Preferably, the second mixture is maintained a sufficient temperature for a sufficient time to obtain (S)-IQL tartrate. One of ordinary skill in the art could easily monitor the reaction to determine when a sufficient amount of time has passed at any given temperature.

Optionally, the process further comprises cooling the second mixture. Preferably, the cooling is to a temperature of about 40° C. to about 0° C., more preferably about 35° C. to about 4° C. or about room temperature, most preferably about 18° C. to about 4° C.

Preferably, the ratio of the organic solvent to water is from about 4:1 to about 1:1 by volume preferably from about 3.5:1 to about 2.3:1 by volume. Preferably, the amount of (D)-tartaric acid is at least about 1 molar equivalent to the amount of IQL. Optionally, the amount of (D)-tartaric acid is about 1 molar equivalent to the amount of IQL.

Optionally, the process further comprises recovering (S)-IQL tartrate from the mixture, such as by precipitating (S)-IQL. Optionally, the precipitating step comprises seeding with (S)-IQL tartrate. Preferably, the seeding takes place during the optional cooling step.

Optionally, the process further comprises filtering, drying, and/or washing the precipitated (S)-IQL tartrate. Preferably, the washing is with a wash solution comprising IPA. Preferably, the drying is carried out at a temperature of about 40° C. to about 60° C. Preferably, the drying is carried out under a pressure of less than one atmosphere or under vacuum.

The present invention further provides a process for preparing (S)-IQL tartrate comprising (a) combining IQL oxalate, water, an organic solvent selected from THF and EtOAc, and an inorganic base to obtain (S)-IQL; and (b) combining the (S)-IQL from step (a) with (D)-tartaric acid to obtain (S)-IQL tartrate. Optionally, the water is added separately or as part of an aqueous solution of the base.

Optionally, the IQL oxalate and the organic solvent are combined prior to the addition of the base. Optionally, water is added prior to the addition of the base. Preferably, the mixture comprising the IQL oxalate and the organic solvent is stirred, preferably at room temperature.

Preferably, the base is added to obtain a pH of from about 10 to about 14, more preferably from about 8 to about 14.

Preferably, the base is selected from the group consisting of KOH, NaHCO₃, KHCO₃, Na₂CO₃, K₂CO₃, and NaOH. Optionally, the base is added as an aqueous solution. Optionally, the base is added dropwise.

Optionally, after the addition of the base, the salts generated are removed, preferably by filtration. Optionally, the salts are washed with the organic solvent. Preferably, the organic solvent after washing is combined with the filtrate.

Optionally, step (a) results in a multi-phase system including an organic phase containing (S)-IQL tartrate. Optionally, an organic solvent selected from C₁-C₄ alcohol and mixtures thereof is added to the organic phase. Preferably, the organic phase contains THF. Preferably, the C₁-C₄ alcohol is ethanol. Preferably, the addition is at about room temperature, more preferably at about 17° C. to about 25° C.

Optionally, after the (D)-tartaric acid addition, a slurry is obtained. Optionally, the slurry is stirred. Preferably, the stirring is for about 0.5 hours to about 24 hours, more preferably for about 1 hour to about 8 hours.

Optionally, the process further comprises recovering the (S)-IQL tartrate obtained. Preferably, the recovery comprises filtering, drying, and washing (S)-IQL tartrate. Preferably, the drying is carried out at a temperature of about 40° C. to about 0° C. Preferably, the drying is carried out under a pressure of less than one atmosphere, more preferably under vacuum.

Optionally, the (S)-IQL tartrate obtained through the processes of the present invention has an enantiomeric purity of at least about 90%, more preferably at least about 95%. Optionally, when water and IPA are used, the (S)-IQL tartrate obtained has an enantiomeric purity of at least about 98%.

The present invention further provides a process of preparing solifenacin succinate by converting (S)-IQL tartrate made by a process as described above to solifenacin succinate. The conversion may be carried out with or without recovery of the (S)-IQL tartrate.

(S)-IQL tartrate may be converted to (S)-IQL by adding a base, for example, according to the methods disclosed in U.S. patent application No. 60/859,952 or in Naito et al. in J. Med. Chem. 48(21): 6597-6606 (2005), which are incorporated herein by reference.

(S)-IQL may be converted to (S)-IQL alkyl carbamate by reacting with an alkyl carbamate, for example, according to the methods disclosed in U.S. patent application Ser. No. 11/645,021, which is incorporated herein by reference. (S)-IQL alkyl carbamate may be converted to solifenacin by reacting with 3(R)-quinuclidinol in the presence of base, for example, according to the methods disclosed in U.S. patent application No. 60/930,391 and U.S. patent application Ser. No. 11/645,021.

Solifenacin may be converted to solifenacin succinate by reacting with succinic acid, for example, according to the methods disclosed in U.S. patent application No. 60/930,391 and U.S. patent application Ser. No. 11/645,021.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way.

### EXAMPLES

### Example 1

A round bottom flask was loaded with IQL (50 g), IPA (350 ml), and water (150 ml). The mixture was heated to 60° C. for dissolution. Then D-tartaric acid (36 g) was added, and the solution was cooled to 25° C. The product was isolated after 2.5 hours by vacuum filtration, washed with IPA (2×50 ml), dried in a vacuum oven at 50° C. over the weekend to obtain (S)-IQL tartrate (33.5 g, 80% yield, 100% enantiomeric purity).

### Example 2

A round bottom flask was loaded ask was loaded with IQL (10 g), IPA, and water. The mixture was heated to 60° C. for dissolution. Then D-tartaric acid was added, and the solution was cooled and stirred. Where applicable, seeding was performed during the cooling step. The product was isolated by vacuum filtration, washed with a mixture of water and IPA, and dried in vacuum oven at 50° C. over the

TABLE 1

| Tartaric | | | | | Stirring | | | |
|---|---|---|---|---|---|---|---|---|
| acid | | | Acid | | time | | | |
| (molar | IPA | H₂O | addition | | after | Cooling | | |
| equiv. | (ml/g | (ml/g | temp. | | cooling | temp. | Enantiomeric | Yield |
| to IQL) | of IQL) | of IQL) | (° C.) | Seeding | (hrs) | (° C.) | Purity | (%) |
| 1 | 7 | 3 | 60 | − | 2.5 | RT | 98.4 | 83.5 |
| 1 | 6 | 3 | 60 | − | 2.5 | RT | 98.6 | 81.4 |
| 1 | 7 | 2 | 60 | − | 2.5 | RT | 98.8 | 87.1 |
| 1 | 7 | 4 | 60 | − | 2.5 | RT | 97.7 | 75.5 |
| 1 | 5.6 | 2.4 | 60 | − | 2.5 | RT | 98.2 | 85.0 |
| 1 | 8.4 | 3.6 | 60 | − | 2.5 | RT | 99.8 | 77.1 |
| 1 | 7 | 3 | 40 | − | 2.5 | RT | 98.1 | 80.0 |
| 1 | 7 | 3 | 25 | − | 2.5 | RT | 97.6 | 79.0 |
| 1 | 7 | 3 | 60 | + | 2.5 | RT | 98.7 | 77.9 |
| 1 | 7 | 3 | 60 | − | 5 | RT | 98.7 | 79.2 |
| 1 | 7 | 3 | 60 | − | 15 | RT | 89.9 | 85.4 |
| 1 | 7 | 3 | 60 | − | 2.5 | 15° C. | 99.6 | 91.8 |
| 1 | 7 | 3 | 60 | − | 2.5 | 5° C. | 99.5 | 92.0 |

weekend to obtain (S)-IQL tartrate. The experiments and results are summarized in Table 1.

### Example 3

A round bottom flask was loaded with IQL (50 g), EtOAc (350 ml), and water (150 ml). The mixture was heated to 60° C. for dissolution. Then D-tartaric acid (36 g) was added, and the solution was cooled to 25° C. The product was isolated after 1.5 hours by vacuum filtration, washed with EtOAc (2×50 ml), dried in vacuum oven at 50° C. overnight to obtain (S)-IQL tartrate (37.25 g, 89% yield, 94.5% enantiomeric purity).

### Example 4

A mixture of IQL oxalate (100 g), THF (500 ml), and water was stirred at RT. NaOH solution (47%, 50 ml) was added dropwise (pH=14), and the salts were removed by filtration and washed with THF (100 ml). The combined filtrate layers were separated. Absolute EtOH (500 ml) was added to the organic phase at RT, and then D-tartaric acid (50 g) was added. A slurry was obtained within 5 min, and stirred for 3.75 hr at RT. The product was isolated by vacuum filtration, washed with EtOH (2×100 ml), and dried in vacuum oven at 50° C. over night to obtain (S)-IQL tartrate (50.05 g, 83.4% yield, 98.3% enantiomeric purity).

### Example 5

A 500 ml round bottom flask was loaded with IQL oxalate (10 g) and EtOAc (100 ml), and stirred at RT. NaOH solution (2M, 250 ml) was added dropwise (pH=14), and the salts were removed by filtration. The filtrate layers were separated, and D-tartaric acid (5 g) was added to the organic phase. Slurry was obtained within 5 min, and stirred for 1 hr at RT. The product was isolated by vacuum filtration, washed with EtOH (2×10 ml), and dried to obtain (S)-IQL tartrate (5.44 g, 90.6% yield, 93.4% enantiomeric purity).

1. A process for preparing 1(S)-phenyl-1,2,3,4-tetrahydroisoquinoline tartrate, comprising combining 1-phenyl-1,2,3,4-tetrahydroisoquinoline, (D)-tartaric acid, and an organic solvent selected from isopropanol and ethyl acetate.

2. The process of claim 1, wherein
(a) the 1-phenyl-1,2,3,4-tetrahydroisoquinoline and the organic solvent is combined with water to form a first mixture; and
(b) the (D)-tartaric acid is combined with the first mixture of step (a) to form a second mixture containing 1(S)-phenyl-1,2,3,4-tetrahydroisoquinoline tartrate.

3. The process of claim 2, where in the ratio of the organic solvent to water is from about 4:1 to about 1:1 by volume.

4. The process of claim 2, further comprising heating the first mixture of step (a) before the addition of the (D)-tartaric acid and heating the second mixture of step (b).

5. The process of claim 4, wherein the second mixture is heated to a temperature of about 40° C. to about reflux temperature.

6. The process of claim 5, wherein the second mixture is heated to a temperature of about 40° C. to about 65° C.

7. The process of claim 2, wherein the wherein the second mixture is maintained at a sufficient temperature for a sufficient time to obtain 1(S)-phenyl-1,2,3,4-tetrahydroisoquinoline tartrate.

8. The process of claim 4, further comprising cooling the second mixture.

9. The process of claim 8, wherein cooling is to a temperature of about 40° C. to about 0° C.

10. The process of claim 9, wherein cooling is to a temperature of about 35° C. to about 4° C.

11. The process of claim 10, wherein cooling is to a temperature of about 15° C. to about 5° C.

12. The process of claim 1, further comprising recovering 1(S)-phenyl-1,2,3,4-tetrahydroisoquinoline tartrate.

13. The process according to claim 12, wherein the recovery comprises precipitating 1(S)-phenyl-1,2,3,4-tetrahydroisoquinoline tartrate, filtering the precipitate, washing the precipitate, and drying the precipitate.

14. The process according to claim 13, wherein the washing is with a wash solution comprising isopropanol.

15. A process for the optical resolution of 1-phenyl-1,2,3,4-tetrahydroisoquinoline through preparing 1(S)-phenyl-1,2,3,4-tetrahydroisoquinoline tartrate, comprising:
(a) combining 1-phenyl-1,2,3,4-tetrahydroisoquinoline oxalate, water, an organic solvent selected from tetrahydrofuran and ethyl acetate, and an inorganic base to obtain 1(S)-phenyl-1,2,3,4-tetrahydroisoquinoline; and
(b) combining the 1(S)-phenyl-1,2,3,4-tetrahydroisoquinoline from step (a) with (D)-tartaric acid to obtain 1(S)-phenyl-1,2,3,4-tetrahydroisoquinoline tartrate.

16. The process of claim 15, wherein the base is added in a sufficient amount to obtain a pH of from about 10 to about 14.

17. The process of claim 16, wherein the base is added in a sufficient amount to obtain a pH of about 8 to about 14.

18. The process of claim 15, wherein the base is selected from the group consisting of KOH, NaHCO₃, KHCO₃, Na₂CO₃, K₂CO₃, and NaOH.

19. The process of claim 18, wherein the base is NaOH.

20. The process of claim 15, wherein step (a) results in a multi-phase system including an organic phase containing 1(S)-phenyl-1,2,3,4-tetrahydroisoquinoline, and wherein a C₁-C₄ alcohol is added to the organic phase.

21. The process of claim 20, wherein the C₁-C₄ alcohol is ethanol.

22. The process of claim 20, wherein the alcohol is added at room temperature.

23. The process of claim 15, wherein a slurry is obtained after the addition of (D)-tartaric acid.

24. The process of claim 23, wherein the slurry is stirred.

25. The process of claim 24, wherein the stirring about 0.5 hours to about 24 hours.

26. The process of claim 25, wherein the stirring about 1 hours to about 8 hours.

27. The process of claim 15, further comprising recovering 1(S)-phenyl-1,2,3,4-tetrahydroisoquinoline tartrate.

28. The process of claim 1, wherein the enantiomeric purity of the 1(S)-phenyl-1,2,3,4-tetrahydroisoquinoline tartrate is at least about 90%.

29. The process of claim 28, wherein the enantiomeric purity of the 1(S)-phenyl-1,2,3,4-tetrahydroisoquinoline tartrate is at least about 95%.

30. The process of claim 29, wherein the enantiomeric purity of the 1(S)-phenyl-1,2,3,4-tetrahydroisoquinoline tartrate is at least about 98%, and wherein the organic solvent used is isopropanol.

31. A process of preparing solifenacin or solifenacin succinate, comprising converting the 1(S)-phenyl-1,2,3,4-tetrahydroisoquinoline tartrate prepared according to claim 1 to solifenacin or solifenacin succinate.

32. (canceled)

33. The process of claim 15, wherein the enantiomeric purity of the 1(S)-phenyl-1,2,3,4-tetrahydroisoquinoline tartrate is at least about 90%.

34. The process of claim 33, wherein the enantiomeric purity of the 1(S)-phenyl-1,2,3,4-tetrahydroisoquinoline tartrate is at least about 95%.

35. The process of claim 34, wherein the enantiomeric purity of the 1(S)-phenyl-1,2,3,4-tetrahydroisoquinoline tartrate is at least about 98%, and wherein the organic solvent used is tetrahydrofuran.

36. A process of preparing solifenacin or solifenacin succinate, comprising converting the 1(S)-phenyl-1,2,3,4-tetrahydroisoquinoline tartrate prepared according to claim 15 to solifenacin or solifenacin succinate.
